(19) United States
(12) Patent Application Publication
(10) Pub. No.: US 2008/0003629 A1
(43) Pub. Date: Jan. 3, 2008

(54) Device and method for detection of vitamins and nutritional minerals

(76) Inventor: Shayne Kenneth Morris, South Ogden, UT (US)

Correspondence Address: Zachary Miles, Esq, 4072 Skyline, Ogden, UT, 84403, US

(21) Appl. No.: 11/784,646

(22) Filed: Apr. 9, 2007

Related U.S. Application Data
(60) Provisional application No. 60/790,114, filed on Apr. 7, 2006.

(51) Int. Cl. C12Q 1/48; C12Q 1/26; C12Q 1/32; C12M 3/00

(52) U.S. Cl. 435/15; 435/25; 435/26; 435/287.2

(57) ABSTRACT

A novel test device, composition and method used for determining the presence of vitamins and/or minerals in a test sample. The device comprises a carrier matrix incorporated with separate compositions for the detection of individual vitamins and/or minerals.

### CROSS-REFERENCE TO RELATED APPLICATIONS

This application claims the benefit of U.S. Provisional Patent Application No. 60/790,114, filed Apr. 7, 2006, the entirety of which is incorporated herein by reference.

### TECHNICAL FIELD

The present disclosure relates broadly to novel screening methods and systems for detecting and quantifying vitamins and nutritionally important minerals, for example, analyte(s) or ligand(s).

### BACKGROUND

There are numerous reasons to detect and quantify levels of vitamins and minerals. Medically, physicians may choose, for example, to monitor vitamin and mineral levels within an individual to detect various disease states, therapy progression, and overall physical health and condition. Moreover, being able to monitor the vitamin and nutritional mineral status rapidly and over a period of time is paramount to a persons overall nutritional status. Current methods of sampling followed by laboratory analyses provide only a snapshot of the individual's nutritional status.

Test devices in the form of test strips and similar rapid elements are used in the analysis of various types of samples. Their popularity stems from, among other reasons, their convenience and speed. As a result, rapid systems have been designed for detecting various significant substances like glucose and cholesterol in blood or urine. They have proven to be extremely advantageous in assisting the diagnosis and treatment of disease states in humans and animals.

Commercially, the detection of vitamins and minerals confirms potency in a given supplement, or, as previously stated, provides guidance on which supplement, vitamin, or mineral is deficient within an individual or a disease state that may be rectified by the addition of a vitamin or mineral. Additionally, a supplement may be given to reduce a compound, agent, sample, or condition.

Existing vitamin and mineral testing technologies require laboratories staffed with trained chemists and specific testing methodologies (AOAC, USP). Furthermore, tests performed in the laboratory are expensive and time consuming. Therefore, what is needed is a simple method of measuring vitamins and minerals. Currently, there are no affordable, sensitive, reliable, versatile, and easy methodologies capable of detecting vitamins and minerals.

### SUMMARY

The present invention provides novel systems, devices, and methods for identifying and quantifying vitamin and mineral content. Examples of vitamins and minerals detectable by the systems, devices and methods disclosed herein include, but are not limited to ascorbic acid, niacin, thiamine, riboflavin, nicotinic acid, pyridoxine, biotin, vitamin B₁₂, folic acid, Calcium, Magnesium, Zinc, Iron and Copper.

In one embodiment, the system includes an indicator compound, enhancer substance, buffer compound, and anti-interference substance.

In another embodiment, a spectrophotometer is used for quantification of the vitamins and minerals present in a sample such as urine through measurement of the device.

In another embodiment, the system can be utilized to monitor the vitamin and mineral levels of an individual. For example, the disclosed systems may be used to measure deficiencies of a particular vitamin or mineral such that an appropriate remedy could be administered to rectify the deficiency.

In yet another embodiment, the system may be used to test industrial chemicals, household chemicals, pesticides, fungicides, fertilizers and the like for a given vitamin or mineral content. A test of this type could provide scientific evidence regarding whether or not specific vitamins or minerals are contained in the aforementioned compounds and their distribution in the environment.

In another embodiment, the system may be used in the pharmaceutical and supplement industry to screen new vitamins and minerals.

In still another embodiment, the system provides for monitoring of vitamins and minerals (found in physiological forms, such as thiamine (Cocarboxylase)) in serum, saline, urine or any other appropriate medium.

Many alterations and variations of the systems, methods and devices exist as described herein. The elements necessary to carry out the methods and produce the compositions and devices as herein disclosed can be adapted for application in any test medium, whether varying in size, shape, or sample. The disclosure further provides a general method for detecting vitamins and minerals.

### BRIEF DESCRIPTION OF THE DRAWINGS

The following drawings form part of the present specification and are included to further demonstrate certain embodiments. These embodiments may be better understood by reference to one or more of these drawings in combination with the detailed description of specific embodiments presented herein.

FIG. 1 illustrates a schematic test strip according to one embodiment.

FIG. 2 illustrates a schematic test strip according to another embodiment.

FIG. 3 shows the use of the test strip in a sample, and includes an example assay response, quantitative and qualitative, to riboflavin.

FIG. 4 shows the use of a spectrophotometer to quantitatively analyze the sample improving accuracy and efficiency.

### DEFINITIONS

It is also to be understood that the terminology used herein is used for the purpose of describing particular embodiments only. The terms are not intended to be limiting because the scope of the present invention is intended to be defined by the appended claims and equivalents thereof.

As used in this specification and the appended claims, the singular forms “a”, “an” and “the” include plural referents unless the content clearly dictates otherwise.

The term “about” when referring to a numerical value or range is intended to encompass the values resulting from experimental error that can occur when taking measurements.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a weight range of about 1 wt % to about 20 wt % should be interpreted to include not only the explicitly recited concentration limits of 1 wt % to about 20 wt %, but also to include individual concentrations such as 2 wt %, 3 wt %, 4 wt %, and sub-ranges such as 5 wt % to 15 wt %, 10 wt % to 20 wt %, etc.

### DETAILED DESCRIPTION

The present invention relates to methods, systems, and devices for detecting vitamins and other minerals. In contrast to previously known methods for vitamin and mineral detection, the embodiments disclosed are able to quantitatively and qualitatively detect one or more vitamins or minerals in a given sample in an affordable, sensitive, reliable, versatile, and easy to use way.

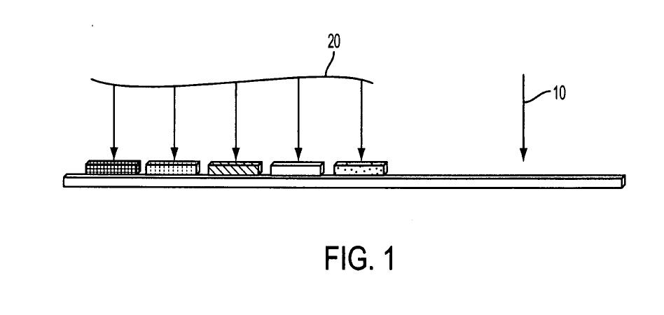

Referring now to FIG. 1, a particular embodiment of the system includes at least one carrier matrix 20 attached to a test strip 10. The carrier matrix 20 may contain any one, any combination, or all of the following: at least one indicator compound, at least one enhancer substance, at least one buffer compound, and at least one anti-interference substance.

Some examples of indicator compounds that may be included in a carrier matrix 20 include but are not limited to methanolic 1-chloro-2,4-dinitrobenze, 2,6-dichloroquinone-4-chloroimide, 2,6-dichloroindophenol, 4-hydrainobenzensulfonic acid, o-phenylenediamine, 4-methylaminophenol sulfate, 2,4-dinitrophenylhydrazine, 5,4-dimethylamino benzylidine rhodamine, 2,2-bipyridine, 2,4,6-tri-2-pyrenidyl-1,3,5-triazindine (TPTZ), p-dimethylaminocinnamaldehyde, phosphomolybdic acid, glyoxyl-bis-2-hydroxanil, nitroprusside sodium, diphenylcarbazide, thiazole yellow, 4,4-nitrophenyl-1-napthol, potasium thiocyanate, glutathione, para-amino salicylic acid, N,N-dichlorohexyldibodiimide, and derivatives of the foregoing.

Additional examples of indicator compounds that may be included in a carrier matrix 20 include compounds selected from the group of enzymes including but not limited to folic acid oxidase, folic acid reductase, horseradish peroxidases, dihydrofolate reductase, riboflavin phosphotransferase, adonsyl transferase, pyridoxine dehydrogenase, thiamine oxidase, NADH oxidase, cobalamin reductase, Acyl coenzyme A oxidase, and nitrite reductase, and derivatives of the foregoing. An oxidase may be coupled to a peroxidase.

Additional examples of indicator compounds that may be included in a carrier matrix 20 include a group of binding molecules including but not limited to riboflavin binding protein, intrinsic factor, streptavidin, avidin and anti-vitamin antibodies and derivatives of the foregoing.

Some examples of enhancer substances that may be included in a carrier matrix 20 include but are not limited to H₂SO₄, NaOH, fluorescein, diazonium salt coupling agents, or oxidases, reducing agents and a wetting agent like polyvinyl alcohol(PVA), hydroxypropyl cellulose (HPC), guar gum, other polysaccharides, thiochrome, paraffin, sodium acetate, high molecular weight polysaccharides, albumin, dioctyl sulfosuccinate sodium, trichloroacetate (TCA), polyvinylpyrrolidone (PVP) or sodium dodecyl sulfate, and derivatives of the foregoing.

Some examples of buffer compounds that may be included in a carrier matrix 20 include but are not limited to HEPES, TRIS, Trizma maleate, citrate, succinate, borate, and Phosphate, and derivatives of the foregoing. These buffers may create a pH range of approximately 4.5 to 7.5, however greater pH ranges may be achieved.

Some examples of anti-interference substances that may be included in a carrier matrix 20 include but are not limited to Ethyldiamino-tetra-acetic acid (EDTA), Iron hydroxyethyl ethylenediamin triacetic acid (FeHEDTA), ascorbic oxidase, and ascorbic acid, and derivatives of the foregoing.

In an exemplary embodiment, an individual carrier matrix 20 containing an indicator compound is formed by impregnating a pad, such as of cellulose, PTFE, Nylon, silica or any other suitable material, with an indicator compound and placing the resulting carrier matrix 20, removably or permanently, on a test strip 10. Alternatively, an individual carrier matrix 20 is formed by impregnating a pad with at least one, or a combination, of the following: at least one enhancer substance, at least one indicator compound, at least one buffer compound, and at least one anti-interference substance.

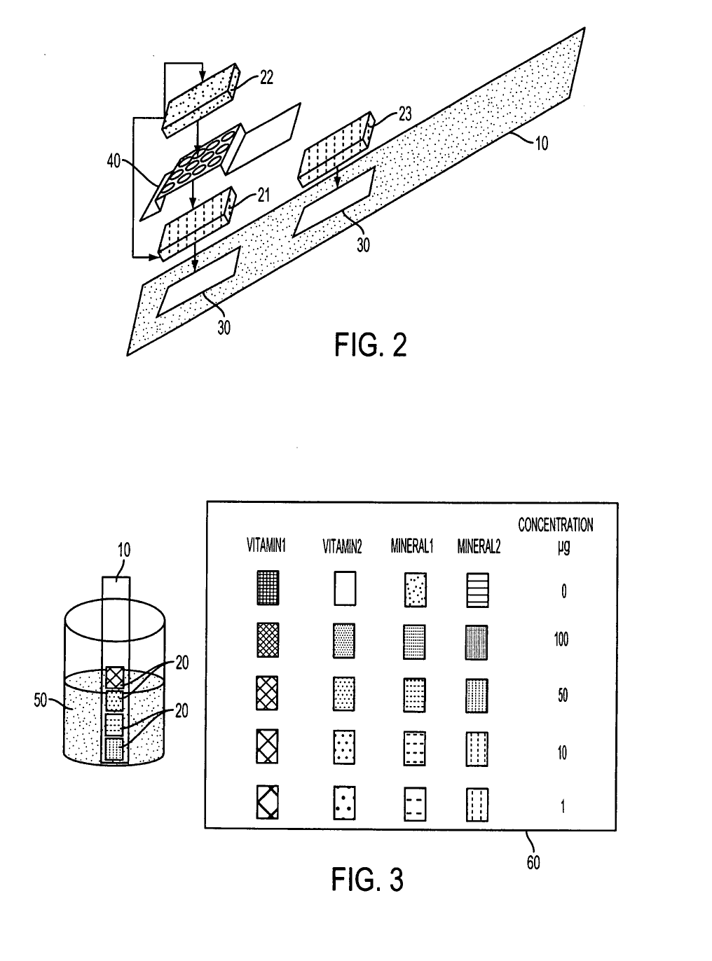

Referring now to FIG. 2, in another embodiment, and by way of example, the test strip 10 is approximately 1 cm wide by 11 cm long (this dimension may vary depending on use) where at least one carrier matrix 20, comprised of a cellulose, PTFE, nylon or silica pad, as way of example 1 cm square (this dimension may vary depending on use) is impregnated with indicator and at least one of the following: buffer, anti-interference substance and enhancer, and attached to a test strip 10. Alternatively, the carrier matrix 20 is formed by impregnating a cellulose, PTFE, nylon or silica pad with at least one, or a combination, of the following; at least one enhancer substance, at least one indicator compound, at least one buffer compound, and at least one anti-interference substance. The carrier matrix 20 containing the impregnated pads may be dried to prevent light and air oxidation. The process of drying the carrier matrix 20 may occur before or after attaching the carrier matrix 20 to the test strip 10. As shown in FIG. 2, the test strip 10 may have a single carrier matrix 20 or multiple carrier matrixes 20 along the test strip 10, two carrier matrixes 20 stacked or multiple carrier matrixes 20 and sets of carrier matrixes 20 stacked along the test strip 10.

In one embodiment, and as shown in FIG. 2, the prepared carrier matrixes 20 are fastened onto the test strip 10 using double-sided adhesive 30 but can also be fastened by hot glue or other adhesives. By way of specific example, and as shown in FIG. 2, a thiamine (Vitamin B₁) test strip 10 is prepared using 3 carrier matrixes 20. The first carrier matrix 21 directly adhered to the test strip 10 has been impregnated with the indicator methanolic 1-chloro-2,4-dinitrobenze. Likewise the second carrier matrix 22 attached to the first carrier matrix 21 contains buffer, EDTA, and wetting agent. The first carrier matrix 21 is fastened to the test strip 10 with double-sided adhesive 30. Likewise the second carrier matrix 22 is attached to the first carrier matrix 21 with double-sided adhesive 40, which may have permeable perforations. The third carrier matrix 23 is attached to the test strip 10 and contains buffer, EDTA, and wetting agent.

Referring now to FIG. 3, the test strip 10 and the carrier matrixes 20 may be placed in contact with a sample 50 wherein the user is enabled to observe any detectable response formed therein.

By way of specific example, and as shown in FIG. 3, the sample 50 may be or contain urine and vitamin 1, riboflavin, pyridoxine or thaiamine. When a vitamin in the sample 50 comes into contact with the carrier matrixes 20 adhered to the test strip 10, wherein the carrier matrixes 20 contain FMN reductase, pyridoxine oxidase, or thiamine oxidase, the reaction produces peroxide, which reacts with horseradish peroxidase giving an observable response. Conventional peroxidase colormetric substrates include but are not limited to: 2,2′-Azino-bis(3-ethylbenzthiazoline-6-sulfonic acid(ABTS), 3,3′,5,5′-Tetramethyl-benzidine(TMB), 5-Aminosalicylic acid and 3-amino-9-ethylcarbazole(AEC), and derivatives of the foregoing, which may be used in this embodiment of the device for both quantitative and qualitative colorimetric development.

The concentration of the analyte or ligand in solution may alter the intensity of the response. Detectable responses can be compared to a series of standardized ligand or analyte concentrations using the same chemical/enzymatic conditions thus providing both a qualitative and semi-quantitative determination.

Development of a visible detection may require additional processing i.e. holding for a specific development time, adding heat, or a secondary reagent using an ultra violet pen to illuminate the sample.

A test could additionally be calibrated. By way of specific example, a Thiamine test could be calibrated as follows:
A. A 0.1 M solution of aqueous TRIS was prepared and adjusted to pH 6.5. Polyvinyl alcohol was diluted in solvent to 0.005% w/w. 1 g of p-aminoacetophenone was dissolved in 15 ml of HCl and diluted to 100 ml with DH₂O. A 5% solution of sodium nitrite is preprared and then combined with the p-aminoacdetophenone and stirred.
B. The first cellulose or silica pad is soaked in part of a solution and dried at 40° C. along with 0.001% FeHEDTA and a wetting agent
C. Next a sodium hydroxide-carbonate 5% solution is prepared. This solution is soaked into a separate cellulose or silica pad.
D. The pad from part C is attached to the test strip and the pad from part B is attached to pad C with adhesive.

A standard curve is created using Thiamine HCl in the following concentrations: (1 mg/ml, 0.25 mg/ml, 0.01 mg/ml, and 0.005 mg/ml).

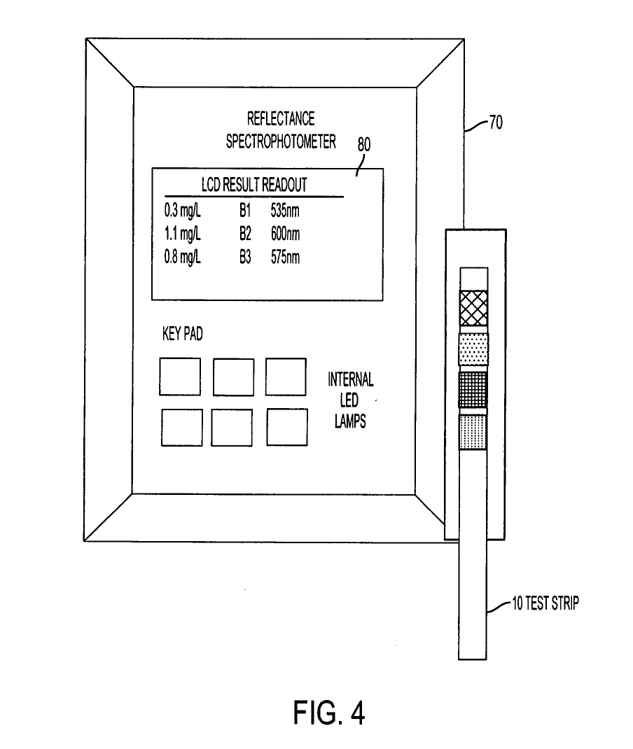

Referring now to FIG. 4, in another embodiment, the system uses a reflectance/transmittance spectrophotometer/photometer 70 to achieve more accurate, efficient and reproducible calorimetric readings 80. The spectrophotometer 70 also allows quantitative evaluation of the device through the detection of specific wavelengths of light. This measurement is accomplished by the insertion of the test strip 10 into the spectrophotometer 70, such that the spectrophotometer can make measurements and provide readings 80. The concentration of the analyte (vitamin or mineral) in the sample is proportional to the intensity and wavelength of color generated.

Quantities of indicator compounds, enhance substances, buffer compounds, anti-interference substances to effectuate the chemical reactions contemplated herein to give the desired result are well known in the art and may be modified to vary time, intensity, or sensitivity depending upon the application of the present invention. As way of example, the following have been used;
(a) chemical indicator compounds −0.02% w/v to 5% w/v
(b) enzymatic indicator compounds 0.5 to 20 mg/ml based on assayed enzymatic activity in units/mg.
(c) enhancer substances −0.01% w/v to 10% w/v
(d) buffer compounds −0.01M to 1.0M.
(e) anti-interference substance −0.001% w/v to 1.0% w/v

It should be emphasized again that given the particular application that variations of the disclosed concentrations may be used.

What is claimed is:

1. A test device for determining the presence of materials in a sample comprising at least one indicator compound attached to a test strip.

2. A test device for determining the presence of vitamins or minerals in a biological sample comprising:
a. at least one indicator compound;
b. at least one enhancer substance;
c. at least one buffer compound; and
d. at least one anti-interference substance.

3. The test device of claim 2 wherein the test device creates detectable analyte responses in the 200-900 nm wavelength ranges.

4. The test device of claim 1 for determining the presence of materials in a sample volume wherein the test device also comprises at least one substance selected from the group consisting of: enhancer substances, buffer compounds, and anti-interference substances.

5. A method for determining the presence of vitamins or minerals in a biological sample comprising the steps of initiating contact between a sample and a test strip containing at least one carrier matrix.

6. The device of claim 1, 2 or 3 wherein the indicator compound is selected from the group consisting of methanolic 1-chloro-2,4-dinitrobenze, 2,6-dichloroquinone-4-chloroimide, 2,6-dichloroindophenol, 4-hydrainobenzensulfonic acid, o-phenylenediamine, 4-methylaminophenol sulfate, 2,4-dinitrophenylhydrazine, 5,4-dimethylamino benzylidine rhodamine, 2,2-bipyridine, 2,4,6-tri-2-pyrenidyl-1,3,5-triazindine (TPTZ), p-dimethylaminocinnamaldehyde, phosphomolybdic acid, glyoxyl-bis-2-hydroxanil, nitroprusside sodium, diphenylcarbazide, thiazole yellow, 4,4-nitrophenyl-1-napthol, potasium thiocyanate, glutathione, para-amino salicylic acid, N,N-dichlorohexyldibodiimide.

7. The device of claim 1, further comprising at least one substance selected from the group consisting of: enhancer substances, buffer compounds, and anti-interference substances.

8. The device of claim 2, 3 or 6 wherein the enhancer substance is selected from the group consisting of: H₂SO₄, NaOH, fluorescein, diazonium salt coupling agents, polyvinyl alcohol(PVA), hydroxypropyl cellulose (HPC), guar gum, thiochrome, polyvinylpyrrolidone (PVP), paraffin, sodium acetate, albumin, dioctyl sulfosuccinate sodium, trichloroacetate (TCA) and sodium dodecyl sulfate.

9. The device of claim 2 or 3 wherein the buffer compound is selected from the group consisting of: HEPES, TRIS, Trizma maleate, citrate, succinate, borate and Phosphate.

10. The device of claim 1, 2 or 3 wherein the indicator compound is impregnated into at least one pad, then the resulting carrier matrix is attached to the test strip.

11. The device of claim 2, 3 or 6 wherein the enhancer substance, buffer compound and anti-interference substance are impregnated into at least one pad, then the resulting carrier matrix is attached to the test strip.

12. The device of claim 9 or 10 wherein the carrier matrix is attached to the test strip with adhesive material.

13. The device of claim 9 or 10 wherein the pad is cellulose, PTFE, nylon or silica.

14. The device of claim 11 or 12 wherein the adhesive material is double sided adhesive.

15. The device of claim 13 wherein the double sided adhesive is permeably perforated.

16. The device of claim 1, 2 or 3 wherein the indicator compound is selected from the group consisting of: folic acid oxidase, folic acid reductase, horseradish peroxidases, dihydrofolate reductase, riboflavin phosphotransferase, adonsyl transferase, pyridoxine dehydrogenase, thiamine oxidase, NADH oxidase, cobalamin reductase, Acyl coenzyme A oxidase, and nitrite reductase.

17. The device of claim 1, 2 or 3 wherein the indicator compound is selected from the group consisting of: riboflavin binding protein, intrinsic factor, streptavidin, avidin and anti-vitamin antibodies.

18. The device of claim 1, 2 or 3 wherein the test strip is polystyrene or polycarbonate.

19. The device of claim 2, 3 or 6 wherein the anti-interference substances are selected from the group consisting of: Ethyldiamino-tetra-acetic acid (EDTA), Iron hydroxyethyl ethylenediamin triacetic acid (FeHEDTA), ascorbic oxidase, ascorbic acid.
